(19) United States
(12) Patent Application Publication
(10) Pub. No.: US 2020/0049908 A1
(43) Pub. Date: Feb. 13, 2020

(54) OPTICAL MODULE AND ENDOSCOPE

(71) Applicant: OLYMPUS CORPORATION, Tokyo (JP)

(72) Inventors: Hiroshi IWAISAKO, Shiojiri-shi (JP); Yusuke NAKAGAWA, Ina-shi (JP)

(73) Assignee: OLYMPUS CORPORATION, Tokyo (JP)

(21) Appl. No.: 16/532,566

(22) Filed: Aug. 6, 2019

Related U.S. Application Data
(63) Continuation of application No. PCT/JP2017/005110, filed on Feb. 13, 2017.

(51) Int. Cl. G02B 6/42; G02B 23/26; A61B 1/00; A61B 1/05

(52) U.S. Cl. CPC G02B 6/4233; G02B 6/4239; G02B 6/4245; A61B 1/051; A61B 1/00165; A61B 1/00126; G02B 23/26

(57) ABSTRACT

An optical module includes: an optical element including a light emitting section which emits a light signal and two external terminals on a light emitting surface; an optical fiber configured to transmit the light signal; a ferrule having an insertion hole into which the optical fiber is inserted; and a wiring board where two connection electrodes provided to a first main surface are respectively bonded to the two external terminals via bumps, and the ferrule is provided to a second main surface, wherein the light emitting surface of the light emitting element is inclined with respect to the first main surface at a predetermined inclination angle based on heights of the bumps.

### CROSS REFERENCE TO RELATED APPLICATION

This application is a continuation application of PCT/JP2017/005110 filed on Feb. 13, 2017, the entire contents of which are incorporated herein by this reference.

### BACKGROUND OF INVENTION

### 1. Field of the Invention

The present invention relates to an optical module including: an optical element which emits or receives a light signal; an optical fiber which transmits the light signal; a ferrule having an insertion hole into which the optical fiber is inserted; and a wiring board where the ferrule is provided to a first main surface, and the optical element is mounted on a second main surface, and also relates to an endoscope which includes the optical module.

### 2. Description of the Related Art

An endoscope includes an image pickup device, such as a CCD, at a distal end portion of an elongated insertion portion having flexibility. In recent years, the use of an image pickup device with a large number of pixels in an endoscope has been contemplated. The use of the image pickup device with a large number of pixels increases an amount of signals transmitted to a signal processing device (processor) from the image pickup device. Accordingly, it is preferable to adopt light signal transmission where light signals are transmitted through a thin optical fiber in place of electric signal transmission where electric signals are transmitted through a metal wire. The light signal transmission uses an E/O type optical module (electricity-light converter) which converts an electric signal into a light signal, and an O/E type optical module (light-electricity converter) which converts a light signal into an electric signal.

In the optical module, the occurrence of multiple reflections between an optical element and an optical fiber causes noises.

Japanese Patent Application Laid-Open Publication No. 2001-281503 discloses an optical module where an optical element is disposed so as to be inclined at a predetermined inclination angle with respect to a bottom surface of a ferrule forming a support member of an optical fiber, thus preventing multiple reflections. In this optical element, an angle holding member having a projecting shape is provided only for allowing the optical element to be disposed in an inclined manner

Japanese Patent Application Laid-Open Publication No. 2013-3250 discloses an optical module where a bottom surface of a ferrule on which an optical element is disposed is formed of an inclined surface, thus preventing multiple reflections.

### SUMMARY OF THE INVENTION

An optical module of an embodiment includes: an optical element including an optically functional region which emits or receives a light signal and two external terminals on a front surface of the optical element; an optical fiber configured to transmit the light signal; a ferrule having an insertion hole, the optical fiber being inserted into the insertion hole; and a wiring board having a first main surface and a second main surface which is disposed on a side opposite to the first main surface, two connection electrodes provided to the first main surface being respectively bonded to the two external terminals of the optical element via bumps, the ferrule being provided to the second main surface, wherein the front surface of the optical element is inclined with respect to the first main surface at a predetermined inclination angle based on heights of the bumps.

An endoscope of another embodiment includes an optical module, the optical module including: an optical element including an optically functional region which emits or receives a light signal and two external terminals on a front surface of the optical element; an optical fiber configured to transmit the light signal; a ferrule having an insertion hole, the optical fiber being inserted into the insertion hole; and a wiring board having a first main surface and a second main surface which is disposed on a side opposite to the first main surface, two connection electrodes provided to the first main surface being respectively bonded to the two external terminals of the optical element via bumps, the ferrule being provided to the second main surface, wherein the front surface of the optical element is inclined with respect to the first main surface at a predetermined inclination angle based on heights of the bumps.

### BRIEF DESCRIPTION OF THE DRAWINGS

FIG. 1 is an exploded view of an optical module of a first embodiment;

FIG. 2 is a cross-sectional view of the optical module of the first embodiment;

FIG. 3 is a top plan view of an optical element of the optical module of the first embodiment;

FIG. 4 is a cross-sectional view of an optical module of a modification 1 of the first embodiment;

FIG. 5 is a cross-sectional view of an optical module of a modification 2 of the first embodiment;

FIG. 6 is a cross-sectional view of an optical module of a modification 3 of the first embodiment;

FIG. 7A is a cross-sectional view of an optical module of a modification 4 of the first embodiment;

FIG. 7B is a cross-sectional view of an optical module of a modification 5 of the first embodiment; and

FIG. 8 is a perspective view of an endoscope of a second embodiment.

### DETAILED DESCRIPTION OF THE PREFERRED EMBODIMENTS

### First Embodiment

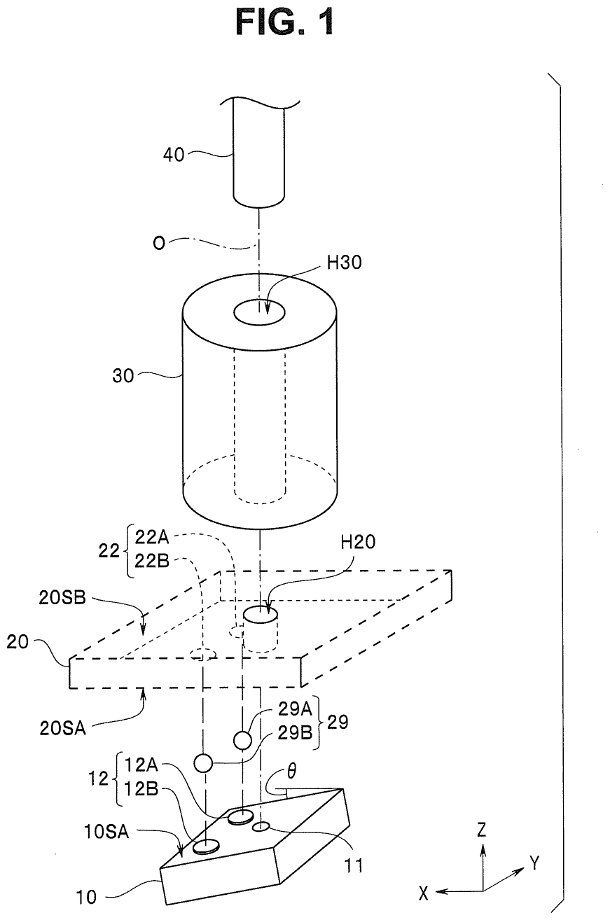

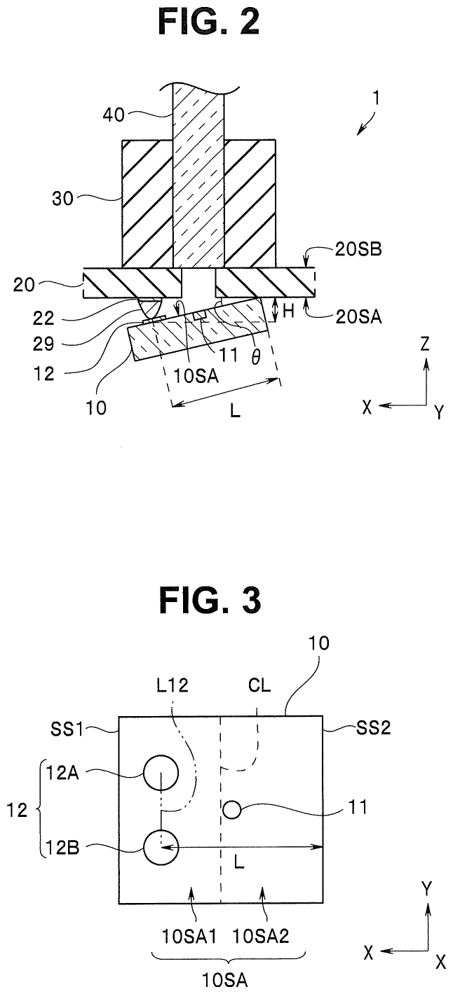

As shown in FIG. 1 and FIG. 2, an optical module 1 of this embodiment includes a light emitting element 10, a wiring board 20, a ferrule 30, and an optical fiber 40.

In the description made hereinafter, drawings referenced in respective embodiments are schematically illustrated. Note that a relationship between a thickness and a width of respective parts, a thickness ratio, a relative angle and the like of each part differ from those of an actual optical module. Some parts may have a different size relationship or a different size ratio between the drawings. Further, some constitutional elements may be omitted from the drawing.

The light emitting element 10 is formed of a VCSEL (vertical cavity surface emitting laser) where a light emitting section 11 is formed on a light emitting surface 10SA forming a front surface of the light emitting element 10. The light emitting section 11 forms an optically functional region which emits a light signal. The light emitting element 10 has an ultra small size of 235 μm×235 μm as viewed in a plan view, for example. The light emitting element 10 includes the light emitting section 11 and two external terminals 12A, 12B on the light emitting surface 10SA. The light emitting section 11 has a diameter of 10 μm. Each of the two external terminals 12A, 12B supplies a drive signal to the light emitting section 11, and has a diameter of 50 μm.

In the case of referring to each of a plurality of constitutional elements having the same function, one letter at the end of a reference symbol may be omitted. For example, each of the external terminals 12A, 12B is referred to as “external terminal 12”.

The wiring board 20 having a flat plate shape has a first main surface 20SA and a second main surface 20SB. The light emitting element 10 is provided on the first main surface 20SA, and the ferrule 30 is provided on the second main surface 20SB. In other words, two connection electrodes 22A, 22B are provided on the first main surface 20SA, and the two connection electrodes 22A, 22B are bonded to the external terminals 12A, 12B of the light emitting element 10 via bumps 29A, 29B. Drive signals are supplied to the connection electrodes 22 through wiring not shown in the drawing. The wiring board 20 may include a processing circuit or the like which converts an image signal from an image pickup device into a drive signal for the light emitting element 10, for example.

The wiring board 20 has a hole portion H20 which forms an optical path for a light signal. The wiring board 20 is an FPC wiring board, a ceramic wiring board, a glass epoxy wiring board, a glass wiring board, a silicon wiring board or the like.

In the case where the wiring board 20 allows light of a light signal to pass therethrough, the hole portion H20 is not required. For example, in the case where infrared light is used for a light signal, provided that an infrared region has high light transmittance, a silicon substrate which is nontransparent in a visible light range may be used as a wiring board having no hole portion H20.

The optical fiber 40 transmits a light signal emitted from the light emitting element 10. The optical fiber 40 includes a core portion which transmits light and which has a diameter of 50 μm, and a clad portion which covers an outer peripheral surface of the core portion, and which has a diameter of 125 μm. An end surface of the optical fiber 40 which forms a light incident surface is orthogonal to an optical axis O.

The ferrule 30 having a columnar shape has an insertion hole H30 which is a through hole into which a distal end portion of the optical fiber 40 is inserted. By inserting the optical fiber 40 into the insertion hole H30, positioning between the light emitting section 11 of the light emitting element 10 and the optical fiber 40 is performed. An inner diameter of the insertion hole H30 may be a columnar shape. Further, provided that the insertion hole H30 can hold the optical fiber 40 with a wall surface of the insertion hole H30, the inner diameter of the insertion hole H30 may be a prism shape, such as a quadrangular prism or a hexagonal prism. A material for forming the ferrule 30 is ceramic, silicon, glass, a metal material, such as stainless steel, or the like. The ferrule 30 may have a substantially rectangular parallelepiped shape, a substantially conical shape or the like.

The ferrule 30 is connected by adhesion to the wiring board 20 by an adhesive agent (not shown in the drawing) in a state where the insertion hole H30 is disposed at a position where the insertion hole H30 opposes the hole portion H20. Note that an inner diameter of the hole portion H20 may be set larger than an outer diameter of the optical fiber 40 so that the hole portion H20 allows insertion of the optical fiber 40.

A distal end surface of the optical fiber 40 having a distal end portion thereof inserted into the insertion hole H30 of the ferrule 30 is parallel to the first main surface 20SA of the wiring board 20. Accordingly, when the light emitting surface 10SA of the light emitting element 10 is provided parallel to the first main surface 20SA of the wiring board 20, there is a possibility that a part of a light signal emitted from the light emitting surface 10SA is reflected off the distal end surface of the optical fiber 40, thus causing multiple reflections.

However, in the optical module 1, the light emitting surface 10SA of the light emitting element 10 is inclined with respect to the first main surface 20SA of the wiring board 20. In other words, the light emitting surface 10SA of the light emitting element 10 and the distal end surface of the optical fiber 40 are not disposed parallel to each other. Multiple reflections are prevented in the optical module 1 and hence, a light signal having low noise and high quality can be transmitted.

As shown in FIG. 3, in the light emitting element 10, an area of the light emitting surface 10SA forming a front surface of the light emitting element 10 is divided into halves, that is, into a first region 10SA1 and a second region 10SA2, by a center line CL. Of the first region 10SA1 and the second region 10SA2, the two external terminals 12A, 12B are provided in the first region 10SA1. It is not necessary that the first region 10SA1 and the second region 10SA2 have completely the same area.

In the case where the two external terminals 12A, 12B are disposed in the light emitting surface 10SA, to prevent short-circuiting or the like, the external terminal 12A is disposed in the first region 10SA1, and the external terminal 12B is disposed in the second region 10SA2 in general. However, the light emitting element 10 is characterized in that the two external terminals 12A, 12B are disposed in an unbalanced manner

The light emitting element 10 has a rectangular parallelepiped shape and hence, as shown in FIG. 3, the light emitting surface 10SA has a rectangular shape. An outer periphery of the light emitting surface 10SA includes a first outer peripheral side SS1 and a second outer peripheral side SS2. The first outer peripheral side SS1 is parallel to an imaginary line L12 connecting the external terminal 12A and the external terminal 12B with each other, and is disposed adjacent to the external terminals 12A, 12B. The second outer peripheral side SS2 is disposed on a side opposite to the first outer peripheral side SS1.

As has already been described, the external terminals 12 are bonded to the connection electrodes 22 of the wiring board 20 via bumps 29. The second outer peripheral side SS2, which forms a portion of the outer periphery of the light emitting surface 10SA, is in contact with the first main surface 20SA of the wiring board 20.

In the case of mounting a light emitting element on a wiring board, three contact points are required so as to unambiguously decide a three-dimensional relative position of the light emitting surface 10SA with respect to the first main surface 20SA. However, only two external terminals 12 are provided to the light emitting element 10, and only two connection electrodes 22 are provided to the wiring board 20. Accordingly, only two bumps 29 are disposed between the light emitting surface 10SA and the first main surface 20SA so that there are only two contact points.

However, the second side SS2 of the light emitting element 10 is in contact with the first main surface 20SA of the wiring board 20 and hence, a relative position of the light emitting surface 10SA with respect to the first main surface 20SA is unambiguously decided.

The light emitting surface 10SA of the light emitting element 10 is inclined with respect to the first main surface 20SA. As shown in FIG. 2, an inclination angle θ is determined by a height H of the bump 29 and a length L from the external terminal 12 forming a bump bonding portion to the second side SS2. In other words, sin θ=H/L is established.

The length L is determined depending on kinds or the like of the light emitting element 10. Accordingly, adjusting the height H of the bump 29 allows an inclination angle to be adjusted to a predetermined inclination angle θ.

For example, to dispose the light emitting element 10 having a length L of 200 μm at an inclination angle θ of 8 degrees, the height H of the bump 29 is set to 28 μm. (Sin (8 degrees)≅0.139≅28/200).

To be more precise, the height H of the bump 29 determining the inclination angle θ is (the height of the bump 29+a thickness of the external terminal 12+a thickness of the connection electrode 22). However, each of the thickness of the external terminal 12 or the thickness of the connection electrode 22 is 0.5 μm to 1 μm, and is extremely smaller than the height H of the bump 29.

The inclination angle θ is set corresponding to specifications of the optical module 1. It is preferable that the inclination angle θ fall within a range of 2 degrees or more and 12 degrees or less, for example. When the inclination angle θ falls within such a range, multiple reflections can be prevented, and there is no possibility that an amount of light reduces significantly.

In the optical module 1, the light emitting surface 10SA can be inclined using the bumps 29 which are essential for supplying a drive signal to the light emitting element 10. Further, the inclination angle θ is set based on the height H of the bump 29.

Accordingly, the optical module has low noise and high productivity.

The bump 29 is a Cu plating bump which is provided to the connection electrode 22 of the wiring board 20 and which is coated with Au. The bump may be provided to the external terminal 12 of the light emitting element 10, or may be provided to the connection electrode 22 and to the external terminal 12.

Alternatively, the bump 29 may be a ball bump disposed between the connection electrode 22 and the external terminal 12. Further, it may be possible to use a stud bump using an Au wire, or a bump made of solder paste or the like.

In the optical module 1, an optical element is the light emitting element 10 which includes the light emitting section 11. However, it goes without saying that even in the case where the optical element is an O/E type optical module which is a light receiving element including a light receiving section, such as a photodiode, as an optically functional region, it is possible to obtain advantageous effects equal to advantageous effects of the optical module 1.

### <Modification of First Embodiment>

Each of optical modules 1A to 1E of modifications of the first embodiment is similar to the optical module 1, thus having the same advantageous effects. Accordingly, constitutional elements having the same functions are given the same reference characters, and the repeated description is omitted.

### <Modification 1>

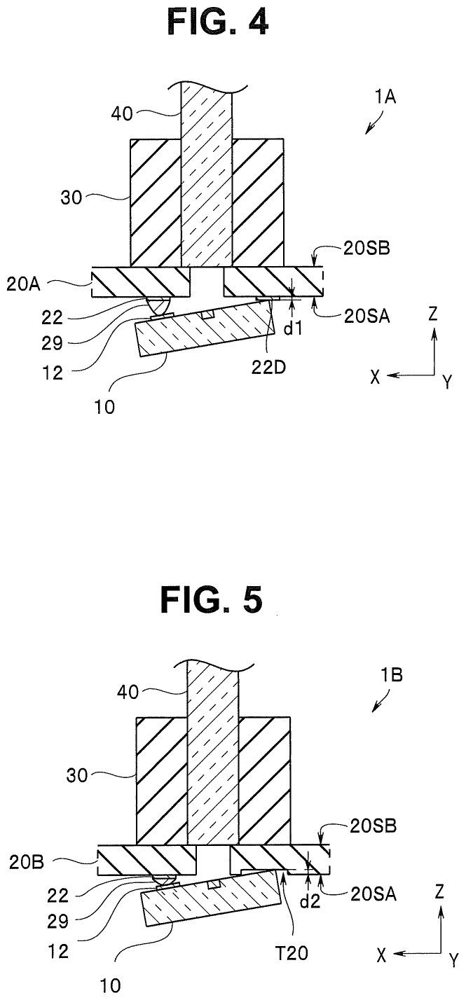

As shown in FIG. 4, in the optical module 1A, a dummy wiring 22D is provided between a first main surface 20SA of a wiring board 20A and an outer periphery of a light emitting surface 10SA of a light emitting element 10. The dummy wiring 22D is a first member which is configured to adjust an inclination angle θ. The dummy wiring 22D, which is an angle adjusting member (first member) having a thickness of dl, is provided simultaneously with the provision of a wiring (not shown in the drawing) provided to the wiring board 20A.

As has already been described, the inclination angle θ can be determined by the height H of the bump 29. However, in the case where limits are imposed on the height H of the bump 29, there is a possibility that an inclination angle cannot be set to a desired inclination angle θ.

However, in the optical module 1A, an inclination angle θ determined by the bump 29 can be further adjusted by the dummy wiring 22D.

The first member is not limited to the dummy wiring made of a conductor provided that the first member is a constitutional element provided at the time of manufacture of the wiring board 20A. The first member may be a resist layer made of a resin, or the dummy wiring may be further covered by a resist.

The first member may be provided to at least any one of spaces selected from a space between the first main surface 20SA and the outer periphery of the light emitting element 10, a space between the external terminal 12 and the bump 29, and a space between the connection electrode 22 and the bump 29.

The first member provided between the external terminal 12 and the bump 29 and the first member provided between the connection electrode 22 and the bump 29 may be formed of any material, such as a metal foil, a metal block, or a metal pole, provided that the material has conductivity. Alternatively, the above-mentioned first members may be formed of an anisotropic conductive material, such as an AFC (anisotropic conductive film) or ACP (anisotropic conductive paste).

A thickness d1 of the first member may be set larger than the height H of the bump 29.

### <Modification 2>

As shown in FIG. 5, in the optical module 1B, a first main surface 20SA of a wiring board 20B has a recessed portion T20 which is configured to adjust an inclination angle θ. The recessed portion T20 is formed by cutting, etching or the like.

In other words, a portion of a light emitting element 10 is inserted into the recessed portion T20. In other words, a second side SS2 of a light emitting element 1 is in contact with a bottom surface of the recessed portion T20.

The wiring board 20B having the recessed portion T20 can adjust an inclination angle θ by changing a depth d2 of the recessed portion T20. Accordingly, the wiring board 20B can be assumed as a member having a function of a first member (angle adjusting member) which can adjust an inclination angle θ.

### <Modification 3>

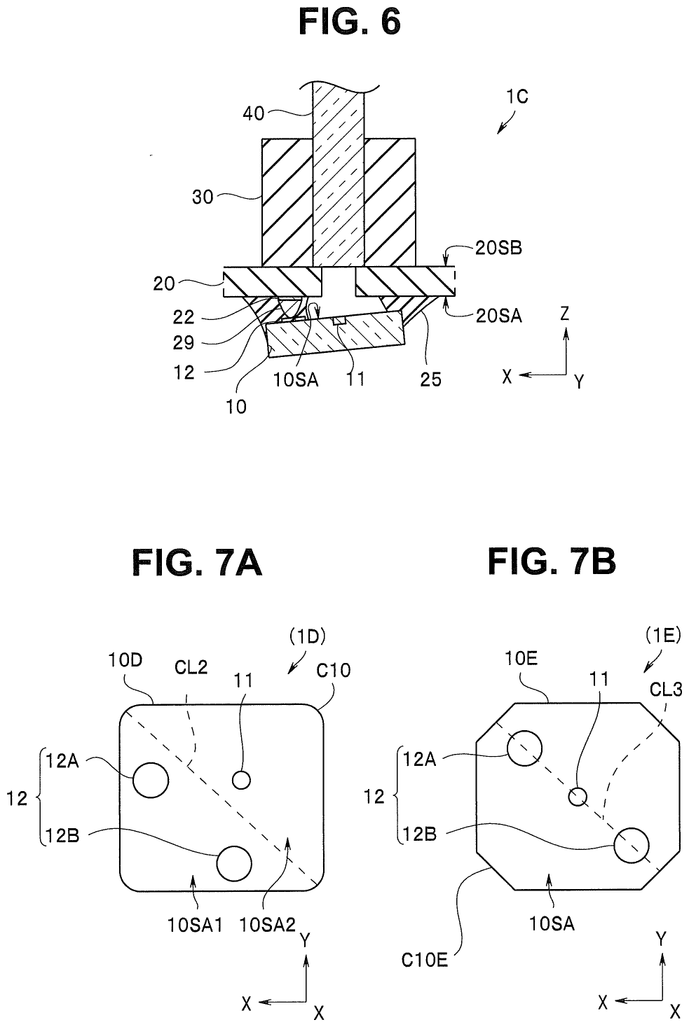

As shown in FIG. 6, in the optical module 1C, a second side SS2 of a light emitting element 10 is not in contact with a first main surface 20SA of a wiring board 20B.

An outer peripheral portion of the light emitting element 10 is fixed to the wiring board 20 by a sealing resin 25 which is a side fill. The sealing resin 25 is made of a resin with excellent moisture resistance, such as an epoxy resin or a silicone resin.

During the manufacture of the optical module 1C, the light emitting element 10 is held by a jig (not shown in the drawing). External terminals 12 of the light emitting element 10 are bonded to connection electrodes 22 of the wiring board 20 via bumps 29. At this point of operation, the light emitting element 10 is held by the jig such that a light emitting surface 10SA has a predetermined inclination angle θ with respect to the first main surface 20SA.

When the bonding is completed, the light emitting element 10 is in a so-called cantilever state so that the second side SS2 is not in contact with the first main surface 20SA of the wiring board 20B. After the external terminals 12 are bonded to the connection electrodes 22 via the bumps 29, a side fill resin in a liquid form is injected into a gap formed between the light emitting surface 10SA of the light emitting element 10 and the first main surface 20SA of the wiring board 20. By performing curing treatment, the side fill resin is formed into the sealing resin 25.

The light emitting surface 10SA is inclined with respect to the first main surface 20SA and hence, a distance of the gap formed between the light emitting surface 10SA and the first main surface 20SA is not uniform. By injecting a resin into the gap from a region having a small distance, due to a surface tension, the outer peripheral portion of the light emitting element 10 is filled with the resin without the resin being entering an optical path region.

In the optical module 1C, an inclination angle θ is determined not only by the height of the bump 29 but also by the adjustment of an angle performed by the jig.

For example, an angle is adjusted while measuring an amount of light and noise of a light signal introduced into an optical fiber 40 so that more appropriate inclination angle θ can be set in the optical module 1C.

It goes without saying that, also in the optical module 1 or the like, the outer peripheral portion of the light emitting element 10 may be fixed to the wiring board 20 by the sealing resin 25 which is a side fill. Further, using a light-shielding resin as a sealing resin can prevent noise caused by external light and leakage of light. An optical path surrounded by the sealing resin 25 may be filled with a refractive index matching material made of a transparent resin.

### <Modification 4>

As shown in FIG. 7A, in the optical module 1D, in the same manner as the optical module 1, an area of a light emitting surface 10SA forming a front surface is divided into halves, that is, into a first region 10SA1 and a second region 10SA2. Of the first region 10SA1 and the second region 10SA2, two external terminals 12A, 12B are provided to the first region 10SA1. However, in a light emitting element 10D, a center line CL2 which divides the area of the light emitting surface 10SA into halves is a diagonal of the light emitting surface 10SA having a substantially rectangular shape.

A corner portion C10 forming a portion of an outer periphery of the second region 10SA2 is in contact with the first light emitting surface 10SA.

### <Modification 5>

As shown in FIG. 7B, in the optical module 1E, the two external terminals 12A, 12B are provided on a diagonal CL3 of a light emitting surface 10SA, having a substantially rectangular shape, so as to be disposed on opposite sides with the light emitting section 11 interposed therebetween.

A portion of an outer periphery of a light emitting element 10 which is in contact with the first light emitting surface 10SA forms an outer periphery of a chamfered corner portion C10E.

In other words, provided that an inclination angle θ can be determined by bumps 29 and a contact portion on the outer periphery of the light emitting surface 10SA, the portion of the outer periphery which is in contact with the first light emitting surface 10SA is not limited to the first outer peripheral side SS1, and may be the outer periphery of the corner portion.

Further, it is not always necessary that the two external terminals are provided only to the first region 10SA1 of the first region 10SA1 and the second region 10SA2 which are obtained by dividing the area of the light emitting surface 10SA into halves.

### Second Embodiment

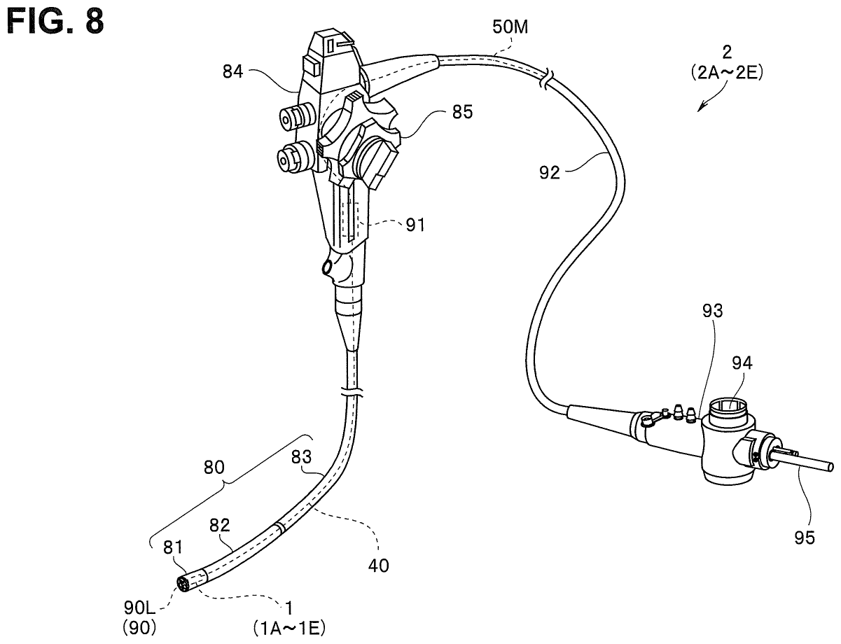

As shown in FIG. 8, an endoscope 2 of this embodiment includes an insertion portion 80, an operation portion 84 provided on a proximal end portion side of the insertion portion 80, a universal cord 92 which is caused to extend from the operation portion 84, and a connector 93 provided on a proximal end portion side of the universal cord 92.

The insertion portion 80 is configured such that a rigid distal end portion 81, a bending portion 82 provided for changing the direction of the distal end portion 81, and an elongated flexible portion 83 having flexibility are successively connected.

An image pickup optical unit 90L, an image pickup device 90, and an E/O type optical module 1 are provided to the distal end portion 81. The E/O type optical module 1 converts an image pickup signal (electric signal) transmitted from the image pickup device 90 into a light signal. The image pickup device 90 is a CMOS (complementary metal oxide semiconductor) image sensor, a CCD (charge coupled device) or the like.

An angle knob 85 and an O/E type optical module 91 are provided to the operation portion 84. The angle knob 85 is provided for operating the bending portion 82. The O/E type optical module 91 converts a light signal into an electric signal. The connector 93 includes an electrical connector portion 94 connected to a processor (not shown in the drawing), and a light guide connecting portion 95 connected to a light source. The light guide connecting portion 95 is connected to an optical fiber bundle which introduces illumination light to the rigid distal end portion 81. The connector 93 may be configured such that the electrical connector portion 94 and the light guide connecting portion 95 are formed into an integral body.

In the endoscope 2, an image pickup signal is converted into a light signal by the E/O type optical module 1 or the like provided to the distal end portion 81, and is transmitted to the operation portion 84 through the thin optical fiber 40 which is allowed to be inserted through the insertion portion 80. The light signal is converted into an electric signal again by the O/E type optical module 91 provided to the operation portion 84, and is transmitted to the electrical connector portion 94 through a metal wire 50M which is allowed to be inserted through the universal cord 92. In other words, a signal is transmitted through the optical fiber 40 in the insertion portion 80 having a small diameter. On the other hand, in the universal cord 92 which is not inserted into the body, thus having an outer diameter where strict limits are not imposed, a signal is transmitted through the metal wire 50M having a diameter larger than that of the optical fiber 40.

In the case where the optical module 91 is disposed in the vicinity of the electrical connector portion 94, the optical fiber 40 may be allowed to be inserted through the universal cord 92 to an area in the vicinity of the electrical connector portion 94. Further, in the case where the optical module 91 is provided to a processor, the optical fiber 40 may be allowed to be inserted to the connector 93.

The endoscope 2 performs light signal transmission where light signals are transmitted through the thin optical fiber 40 in place of electric signal transmission. Accordingly, the insertion portion 80 has a small diameter, thus being minimally invasive.

The present invention is not limited to the above-mentioned embodiments and modifications, and various modifications, combinations and applications are conceivable without departing from the gist of the invention.

What is claimed is:

1. An optical module comprising:
an optical element including an optically functional region which emits or receives a light signal and two external terminals on a front surface of the optical element;
an optical fiber configured to transmit the light signal;
a ferrule having an insertion hole, the optical fiber being inserted into the insertion hole; and
a wiring board having a first main surface and a second main surface, two connection electrodes provided to the first main surface being respectively bonded to the two external terminals of the optical element via bumps, the ferrule being provided to the second main surface, wherein
the front surface of the optical element is inclined with respect to the first main surface at a predetermined inclination angle based on heights of the bumps.

2. The optical module according to claim 1, wherein
a portion of an outer periphery of the front surface of the optical element is in contact with the first main surface of the wiring board.

3. The optical module according to claim 2, wherein
of a first region and a second region obtained by dividing an area of the front surface of the optical element into halves, the two external terminals are provided to the first region, and
a portion of the outer periphery of the second region is in contact with the first main surface.

4. The optical module according to claim 3, wherein
a space between a portion of the outer periphery of the front surface of the optical element which is in contact with the first main surface and the first main surface is filled with a resin.

5. The optical module according to claim 4, wherein
of a first region and a second region obtained by dividing an area of the front surface of the optical element into halves, the two external terminals are provided to the first region, and
a space between a portion of the second region and the first main surface is filled with the resin.

6. The optical module according to claim 2, wherein
a first member having a predetermined thickness is configured to adjust the inclination angle, and the first member is provided to at least any one of spaces selected from a space between the external terminal and the bump, a space between the connection electrode and the bump, and a space between the first main surface and the outer periphery of the optical element.

7. The optical module according to claim 1, wherein
the first main surface of the wiring board has a recessed portion, and a portion of the optical element is inserted into the recessed portion.

8. The optical module according to claim 1, wherein
the predetermined inclination angle is 2 degrees or more and 12 degrees or less.

9. An endoscope comprising an optical module,
the optical module including:
an optical element including an optically functional region which emits or receives a light signal and two external terminals on a front surface of the optical element;
an optical fiber configured to transmit the light signal;
a ferrule having an insertion hole, the optical fiber being inserted into the insertion hole; and
a wiring board having a first main surface and a second main surface, two connection electrodes provided to the first main surface being respectively bonded to the two external terminals of the optical element via bumps, the ferrule being provided to the second main surface, wherein
the front surface of the optical element is inclined with respect to the first main surface at a predetermined inclination angle based on heights of the bumps.
